(19) United States
(12) Patent Application Publication
(10) Pub. No.: US 2023/0046367 A1
(43) Pub. Date: Feb. 16, 2023

(54) SYSTEMS AND METHODS FOR DYNAMICALLY REMOVING TEXT FROM DOCUMENTS

(71) Applicant: Omniscient Neurotechnology Pty Limited, Sydney (AU)

(72) Inventors: Stephane Philippe Doyen, Glebe (AU); Michael Edward Sughrue, Sydney (AU); Dung Nuh To, Belmore (AU)

(21) Appl. No.: 17/399,989

(22) Filed: Aug. 11, 2021

(51) Int. Cl. G06F 40/289; G06F 40/242; G06F 40/166; G16H 10/60; G06K 9/34

(52) U.S. Cl. CPC G06F 40/289; G06F 40/242; G06F 40/166; G16H 10/60; G06K 9/344; G06K 2209/01

(57) ABSTRACT

Disclosed are techniques for building a dynamic dictionary and using the dictionary to remove phrases or words appearing in and out of context in a document. The techniques include, for example, receiving electronic health record (EHR) data, determining, using natural language processing (NLP), an instance of a personal health information (PHI) phrase in the EHR data based on a NLP system confidence metric being above a threshold, determining another instance of the PHI phrase in the EHR data that does not have the same context as the first context, removing the instances of the PHI phrase from the EHR data to produce cleaned EHR data, and taking an action based on the cleaned EHR data. The confidence metric can indicate likelihood that the PHI phrase is a PHI phrase and the metric can be based at least in part on a first context of the PHI phrase.

### TECHNICAL FIELD

The subject matter described herein relates to systems and methods for identifying and removing personal health information (PHI) throughout electronic health record (EHR) data.

### BACKGROUND

EHRs include information about a patient that can be used to provide proper diagnosis and/or treatment to the patient. EHRs can also be used to further medical research efforts. EHRs include information that can be tagged as personal information or PHI. PHI can be a patient's name, disease or diagnosis, medications, and other information personal to the patient. In order to protect patient confidentiality and privacy, PHI can be removed from EHRs before the EHRs are transmitted over networks amongst different devices and/or computing systems, whether the EHRs are used for diagnosis and/or treatment of the particular patient or medical research.

### SUMMARY

Systems and methods described herein are implemented to provide for removal of PHI from EHRs. Compliance requirements often require or encourage removal of PHI from EHRs prior to transmitting such data outside a hospital information technology infrastructure and/or to different devices and computing systems for diagnosis, treatment, and/or medical research. The EHRs can be word documents, scanned PDF documents, other forms of text documents, and/or medical imaging data. The PHI can appear in various locations or contexts in the EHRs. Sometimes, the PHI can appear in atypical locations in the EHRs, such as in a header or footer of a document or overlaid on a patient's body that is represented by the medical imaging data. Although PHI may be useful for identifying the patient and patient-specific treatment, PHI can jeopardize the patient's privacy if the EHRs are exposed to external parties, devices, and/or computing systems. Removal of PHI therefore renders the EHRs as anonymous, which make it difficult for a clinician or other user to identify a patient while reviewing the EHRs. Anonymity therefore preserves patient privacy. Anonymity also provides for use of the EHRs in medical research without compromising or otherwise exposing personal information of the patients that are associated with the EHRs. By preserving privacy, systems and methods described in this specification allow services, e.g., image and other data processing services, in the cloud to facilitate treatment.

A natural language processing (NLP) system can remove PHI from documents by tagging entities in the documents as personal information. These NLP systems can remove PHI from known or typical locations in the documents where the PHI appears. Tagging entities is a form of attributing meanings to words. Tagging can be achieved by using a dictionary or other statistical model that attributes entities based on context (e.g., an entity appears in a text field designated for patient names or an entity follows a prefix such as “Mr.” or “Mrs.”) within the documents. A dictionary, however, can be limited in that it can only be used to tag words or phrases that are already contained in the dictionary. In other words, the NLP systems are limited to removing only the PHI that have been identified and defined in the dictionary.

Statistical models can tag meanings of words or phrases that have not been identified before (e.g., words or phrases that do not appear in a dictionary used by the system), but statistical models can only tag these words when they are presented in context or in conventional/typical locations in documents. Thus, if a new word appears out of context (e.g., the new word does not follow a known prefix), then the statistical models may miss the new word. The new word would not be extracted from the EHR, even if the new word is PHI. Accordingly, patient privacy can be compromised.

Many new words or phrases may need to be tagged in, and subsequently removed from, EHRs in order to preserve patient privacy. These new words can be PHI, such as the patient's name, medication names, disease names, and other personally identifying information. The new words can appear out of context, such as in headers, footers, margins, overlaid on images, or other unconventional locations in EHRs. The disclosed systems and methods provide for expanding dictionaries to facilitate the identification of PHI that has no context or limited context (e.g., does not have a prefix and/or does not have metadata information whether the content is PHI content) and to use the expanded dictionary(ies) to remove PHI in EHRs. As a result, the disclosed systems and methods provide for improved and more accurate identification and removal of PHI from EHRs in order to preserve patient privacy.

Although the disclosed techniques are described in reference to PHI in EHR data, the disclosed techniques can also be applied to other contexts and/or industries. For example, the disclosed techniques can be applied to existing NLP and phrase extraction systems, applications, and/or cloud-based services. The disclosed techniques can also be used in other industries where different forms of data (e.g., documents, image data) include content, e.g., words or phrases, that can be removed.

In one aspect, a method includes receiving electronic health record (EHR) data, determining, using a natural language processing (NLP) system, an instance of a personal health information (PHI) phrase in the received EHR data based at least in part on a NLP system confidence metric being above a threshold, determining another instance of the PHI phrase in the received EHR data, where the another instance of the PHI phrase does not have the same context as the first context, removing the instances of the PHI phrase from the received EHR data to produce cleaned EHR data, and taking an action based on the cleaned EHR data. The confidence metric can indicate the likelihood that the PHI phrase is a PHI phrase and the confidence metric can be based at least in part on a first context of the PHI phrase.

In some implementations, one or more of the following can additionally be implemented either individually or in any feasible combination. For example, a context of the another instance of the PHI phrase can be such that the NLP system confidence metric based on the local context of the another instance is not above the threshold. As another example, the first context of the PHI phrase can include one or more of a group of prefixes including: Mr., Mrs., Ms., Miss, Dr., Doctor, Nurse, Signed by, signed by, Signed electronically by, signed electronically by, Electronically signed by, electronically signed by, Dear, dear, Name, name, NAME, Patient, patient, PATIENT, and RE:. A context of the another instance of the PHI phrase may not include one or more of a group of prefixes including: Mr., Mrs., Ms., Miss, Dr., Doctor, Nurse, Signed by, signed by, Signed electronically by, signed electronically by, Electronically signed by, electronically signed by, Dear, dear, Name, name, NAME, Patient, patient, PATIENT, and RE:. A context of the another instance of the PHI phrase can also include at least one of literals, delimiters, and operators. In some implementations, determining, using a natural language processing (NLP) system, an instance of a personal health information (PHI) phrase can include performing optical character recognition on image data contained in the EHR data. As another example, the PHI phrase can be at least one of a person's names, disease, and medication. As yet another example, the PHI phrase can be a word.

In another aspect, a system can include at least one programmable processor and a machine-readable medium storing instructions that, when executed by the at least one programmable processor, cause the at least one programmable processor to perform operations including receiving EHR data, determining, using an NLP system, an instance of a PHI phrase in the received EHR data based at least in part on a NLP system confidence metric being above a threshold, determining another instance of the PHI phrase in the received EHR data, removing the instances of the PHI phrase from the received EHR data to produce cleaned EHR data, and taking an action based on the cleaned EHR data. The confidence metric can indicate the likelihood that the PHI phrase is a PHI phrase and the confidence metric can be based at least in part on a first context of the PHI phrase. The another instance of the PHI phrase may not have the same context as the first context.

In some implementations, one or more of the following can additionally be implemented either individually or in any feasible combination. For example, a context of the another instance of the PHI phrase can be such that the NLP system confidence metric is not above the threshold. The first context of the PHI phrase can include one or more of a group of prefixes including: Mr., Mrs., Ms., Miss, Dr., Doctor, Nurse, Signed by, signed by, Signed electronically by, signed electronically by, Electronically signed by, electronically signed by, Dear, dear, Name, name, NAME, Patient, patient, PATIENT, and RE:. As another example, a context of the another instance of the PHI phrase may not include one or more of a group of prefixes including: Mr., Mrs., Ms., Miss, Dr., Doctor, Nurse, and Signed by, signed by, Signed electronically by, signed electronically by, Electronically signed by, electronically signed by, Dear, dear, Name, name, NAME, Patient, patient, PATIENT, and RE:. As yet another example, a context of the another instance of the PHI phrase can include at least one of literals, delimiters, and operators. As another example, determining, using an NLP system, an instance of a PHI phrase can include performing optical character recognition on image data contained in the EHR data. Moreover, the PHI phrase can be at least one of a person's names, disease, and medication. As another example, the PHI phrase can be a word.

In another aspect, one or more non-transitory computer program products storing instructions that, when executed by at least one programmable processor, cause the at least one programmable processor to perform operations including receiving EHR data, determining, using an NLP system, an instance of a PHI phrase in the received EHR data based at least in part on a NLP system confidence metric being above a threshold, determining another instance of the PHI phrase in the received EHR data, removing the instances of the PHI phrase from the received EHR data to produce cleaned EHR data, and taking an action based on the cleaned EHR data. The confidence metric can indicate the likelihood that the PHI phrase is a PHI phrase and the confidence metric can be based at least in part on a first context of the PHI phrase. The another instance of the PHI phrase may not have the same context as the first context.

In some implementations, one or more of the following can additionally be implemented either individually or in any feasible combination. For example, a context of the another instance of the PHI phrase can be such that the NLP system confidence metric is not above the threshold. In another example, the PHI phrase can be at least one of a person's names, disease, and medication. Moreover, the first context of the PHI phrase can include one or more of a group of prefixes comprising: Mr., Mrs., Ms., Miss, Dr., Doctor, Nurse, Signed by, signed by, Signed electronically by, signed electronically by, Electronically signed by, electronically signed by, Dear, dear, Name, name, NAME, Patient, patient, PATIENT, and RE:.

In yet another aspect, a method for building a dynamic dictionary can include receiving, by a computing system, data that represents a plurality of EHRs, retrieving, by the computing system and from a data store, machine learning models that were trained to extract first instances of entities in predefined textual locations of the EHRs, extracting, by the computing system and based on applying the machine learning models to the received data, first instances of one or more entities that are in the predefined textual locations in the received data, populating, by the computing system, a dictionary with the one or more extracted entities, determining, by the computing system and based on applying the dictionary to the received data, whether additional instances of the one or more extracted entities are identified in locations that are not predefined textual locations in the received data, extracting, by the computing system and based on determining that the additional instances of the one or more extracted entities are identified in locations that are not predefined textual locations in the received data, the additional instances of the one or more extracted entities, and returning, by the computing system, the received data. In some implementations, the predefined textual locations of the EHRs can include at least one of text boxes, text fields, and signature lines.

The subject matter described herein provides one or more of the following advantages. For example, the disclosed techniques provide for cleaning EHR data so that it can be used by clinicians without compromising on patient privacy or compliance requirements. Clinicians or other medical professionals can therefore use the cleaned EHR data for diagnosis, treatment, and/or medical research. The disclosed techniques enable removal of PHI from all locations in the EHR data, including known or typical locations or contexts as well as unknown or atypical locations or contexts. Some NLP systems may overlook PHI that appears in unknown or atypical locations or contexts in the EHR data. As a result, with some NLP systems, PHI may be removed from some locations in the EHR data but not other, less conventional locations (e.g., headers and/or footers in a document). The techniques disclosed in the specification, on the other hand, can provide for identifying PHI in typical and atypical locations in EHR data such that all instances of PHI can be removed from the EHR data before the EHR data is used for any subsequent processing and/or actions by clinicians or other users. Not only can the disclosed techniques maintain patient privacy and meet compliance requirements, the disclosed techniques can also provide for more EHR data to be accessible and used by clinicians to generate improved diagnosis and treatment of patients and further medical research.

Moreover, the disclosed techniques provide for expanding dictionaries with phrases or words that can be removed from subsequent EHR data. The bigger the dictionaries, the more likely that NLP systems can accurately and quickly identify words or phrases that constitute PHI and ought to be removed from the EHR data.

Additionally, computing systems that implement the disclosed techniques can be continuously trained to identify PHI, using the expanded dictionaries, in locations that are both in and out of context in EHR data. The computing systems can, therefore, more accurately identify and remove PHI wherever it may appear in EHR data. Using the disclosed techniques, PHI phrases or words can be more accurately and quickly removed from EHR data, regardless of whether the PHI were previously known words defined in a dictionary or appearing in context in the EHR data.

Using the techniques described herein, large amounts of EHR data can be processed in little time. For example, the techniques described herein can be performed quickly (e.g., in a matter of seconds). In such limited time, the described techniques can accurately remove PHI from many files or other types of EHR data, including files or other types of EHR data that exceed specified sizes (e.g., MBs). Thus, the disclosed techniques can be used to quickly process batches of EHR data and/or to process EHR data that is large in size.

The details of one or more variations of the subject matter described herein are set forth in the accompanying drawings and the description below. Other features and advantages of the subject matter described herein will be apparent from the description, drawings, and claims.

### BRIEF DESCRIPTION OF DRAWINGS

FIG. 1A is a conceptual diagram of a computing landscape for removing

PHI from EHR data using the disclosed techniques.

FIG. 1B is a block diagram of an NLP deep learning model pipeline used for performing the disclosed techniques.

FIG. 2 illustrates some components of the computing landscape of FIG. 1A that can be used to perform the techniques described herein.

FIG. 3 is a flowchart of a process for removing PHI from EHR data.

FIG. 4 is a flowchart of a process for building a dictionary that can be used to identify and remove PHI from EHR data.

FIG. 5 is a schematic diagram that shows an example of a computing device and a mobile computing device.

Like reference symbols in various drawings indicate like elements.

### DETAILED DESCRIPTION

The disclosed techniques provide for removing PHI from EHR data in order to preserve patient privacy and meet compliance requirements when using EHR data across different computing systems for patient diagnosis, treatment, and/or medical research. Using the techniques described herein, words or phrases, such as names, medications, and/or diseases can be identified and added to one or more dictionaries. The dictionaries can be used to more accurately extract such words from EHR data and other types of documents. More particularly, the dictionaries can be used to extract such words regardless of what context they appear in the EHR data. Sometimes, words can be new, foreign to some languages, or otherwise not known in dictionaries that are used by existing NLP systems. The disclosed techniques, therefore, provide for identifying such words that can appear in different locations in EHR data and removing them. The disclosed techniques can apply to different situations in which contextual clues may be ambiguous and may not provide guidance as to whether the words or phrases are PHI and/or whether they should be removed. Although the document describes the disclosed techniques in reference to PHI in EHR data, the disclosed techniques can also be applied to a variety of other settings and/or situations in which words or phrases can be identified and removed from different forms of data and/or documents.

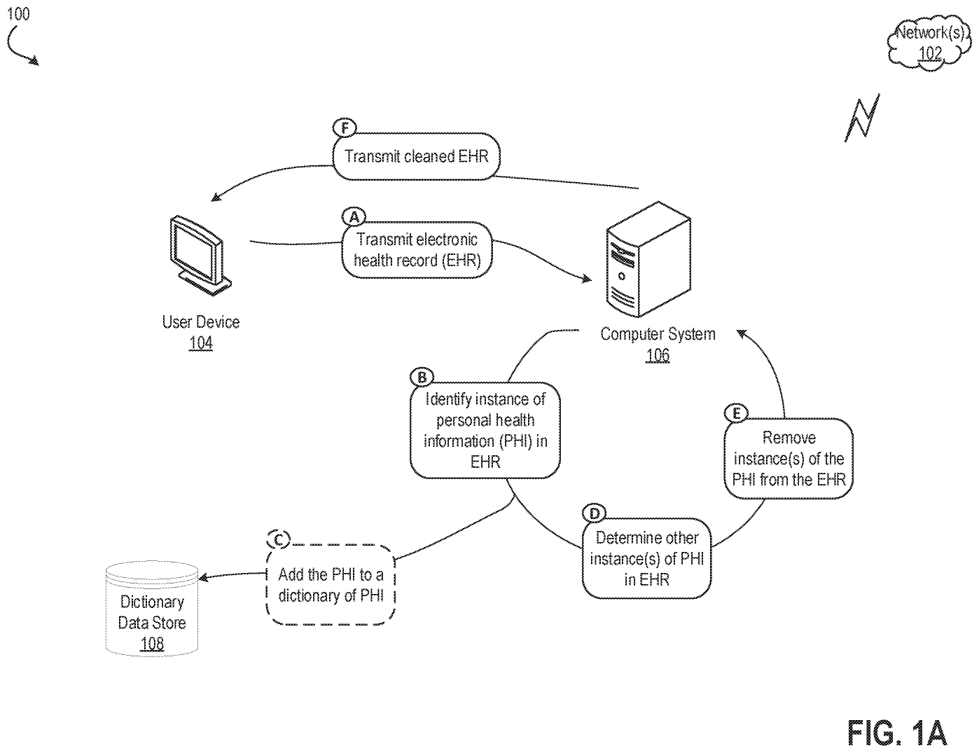

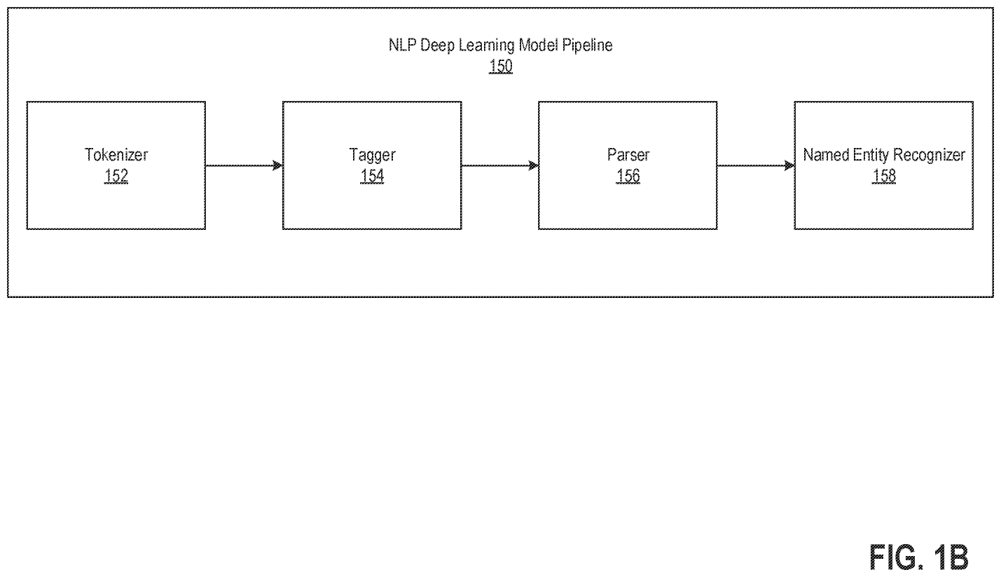

Referring to the figures, FIG. 1A is a conceptual diagram of a computing landscape 100 for removing PHI from EHR data using the disclosed techniques. A user device 104 and a computer system 106 can be in communication (e.g., wired and/or wireless) via network(s) 102. The computer system 106 can also be in communication (e.g., wired and/or wireless) with a dictionary data store 108 via the network(s) 102. The user device 104 can be a computing device such as a mobile device, computer, laptop, tablet, and/or medical device.

The user device 104 can be used by a clinician or other practitioner in a hospital information technology structure. For example, a clinician can collect information about a patient in a hospital or other medical setting. The clinician can complete a medical health form, a type of EHR data, that includes personal information about the patient. The medical health form can be completed electronically at the user device 104 and/or by hand and then scanned at the user device 104. For example, the medical health form can be a scanned PDF document. The personal information can include the patient's name, date of birth, signature, medication names, diagnosis and/or disease names, etc. Other personal information is also possible.

Different types of EHR data can also be received or otherwise created at the user device 104. Sometimes, the EHR data can be medical imaging data. For example, the user device 104 can receive images of the patient's brain from one or more medical imaging devices. The images of the patient's brain can be labeled with personally identifying information, such as the patient's name and/or date of birth. The personally identifying information can be overlaid on the imaging data. As other examples, the medical imaging data can include mammograms, X-rays, Mill scans, CT scans, and any other medical imaging data that can be captured in a medical setting, labeled with personally identifying information, or otherwise attributed to a particular patient.

The EHR data can also be stored in a data store of the hospital or other medical setting and retrieved by the user device 104. The clinician can view the EHR data and/or update the EHR data with PHI or other personal information at the user device 104. The updated EHR data can then be stored in the data store for future use and/or retrieval.

The computer system 106 can be configured to identify and remove PHI from EHR data using the techniques described herein. The computer system 106 can be a cloud computing system. The computer system 106 can also be any other type of computer, computing system, network of computers, and/or network of servers. Sometimes, the computer system 106 can be a same computing system as the user device 104. The computer system 106 can also be part of a same network of hospital information technology structure as the user device 104. In yet some implementations, the computer system 106 can be remote from the user device 104 and/or the hospital information technology structure. A secure connection can be established between the user device 104 and the computer system 106 over the network(s) 102 such that the EHR can be transmitted therebetween without comprising patient privacy.

The user device 104 can transmit EHR data to the computer system 106 in step A. The EHR data can be automatically transmitted to the computer system 106, at predetermined times, and/or based on input from the clinician or other practitioner. In some implementations, the computer system 106 can retrieve or receive the EHR data from the data store instead of the user device 104.

The computer system 106 can identify one or more instances of PHI in the EHR in step B. Using NLP systems and techniques, the computer system 106 can identify PHI that appears in context in the EHR. For example, the computer system 106 can locate patient names, medication names, and/or diseases that appear in conventional or typical fields or locations in the EHR. The conventional or typical fields can include text boxes, text fields, signature lines, and other locations in a document where PHI is typically found. The computer system 106 can also locate patient names, medication names, and/or diseases that appear in a body or main portion of the EHR using contextual clues. The contextual clues can include prefixes, such as Mr., Mrs., Miss, Ms., Dr., and/or Nurse. One or more other contextual clues can be used to locate the PHI in the body or main portion of the EHR, as described throughout this disclosure.

Once the PHI is identified in the EHR, the computer system 106 can optionally add the PHI to a dictionary of PHI in step C. The computer system 106 can add the PHI to the dictionary when the PHI is a name or other word or phrases of words that are not yet known by the computer system 106. For example, the dictionary can include known names such as “Smith” and “John.” However, in step B, the computer system 106 can identify a name such as “Smithkowski,” which follows the prefix “Mr.” but otherwise is not defined in the dictionary with the words “Smith” and “John.” The computer system 106 can identify “Smithkowski” as a new PHI since it appears in the EHR with contextual clues (a prefix that is known to precede names) but has not yet been defined as PHI in the dictionary. Thus, the computer system 106 can add “Smithkowski” to the dictionary of PHI such that the name “Smithkowski” can be identified and extracted (e.g., removed) from subsequent EHR data. In other words, the name “Smithkowski” can be tagged as PHI. During subsequent analysis of EHR data and when the dictionary is applied, the computer system 106 can identify and extract any other instances of “Smithkowski” that appear in the EHR data. By adding names or other PHI to the dictionary, the dictionary can be expanded to encompass a variety PHI that can be more accurately identified and extracted from EHR data.

Adding the PHI to the dictionary can include storing the PHI in the dictionary data store 108. The dictionary data store 108 can securely store one or more dictionaries of PHI. The dictionaries can be used by the computer system 106 to identify PHI in EHR data, whether or not the PHI appears in context or out of context in the EHR data, as described further below. The dictionary data store 108 can also store different types of dictionaries. Each dictionary can be attributed to a different category of PHI. For example, one or more dictionaries can be attributed to patient names. Some dictionaries can be attributed to doctor or other practitioner names. Some dictionaries can include drug or medication names. Some dictionaries can include disease names. Other dictionaries of PHI are also possible.

Next, in step D, the computer system106 can determine other instances of the PHI in the EHR. The computer system 106 can scan through the EHR to determine where the PHI identified in step B may appear again in the EHR. The computer system 106 can be configured to search locations of the EHR that are unconventional locations where the PHI may appear. For example, the computer system 106 can search headers, footers, and/or margins in EHR documents for other instances of the PHI. The computer system 106 can also search a body or main portion of the EHR documents for instances where the PHI may appear without any contextual clues, such as prefixes. The computer system 106 can also search for instances of PHI that may appear on top of portions of image data and/or in metadata of such image data.

As an illustrative example, the name “Smithkowski” can appear in a header on one or more pages of a medical health form. The name “Smithkowski” can also appear in one or more sentences in the body of the medical health form and without the “Mr.” prefix, such as in a sentence handwritten by the clinician. In these examples, the name “Smithkowski” appears out of context in the EHR data. Since the name “Smithkowski” has been previously identified and added to the dictionary as potential PHI content, the computer system 106 can now determine and identify, using the techniques described herein, where the name “Smithkowski” appears in both in context and out of context locations throughout the EHR data.

In scenarios where the name is a common word, all instances of that common word may be removed from the EHR data, regardless of whether the common word appears in the context of a name or another context. For example, if the word “Brain” appears in context with a prefix “Mr.,” the computer system 106 can determine that Mr. Brain is in fact a person, and “Brain” is the person's name. Accordingly, any mention of “Brain” in the EHR data will be removed, even if the mention of “Brain” is in a common context (e.g., text saying “Our brain consists of grey and white matter”) rather than a name context (e.g., text saying “Mr. Brain's visit”). Removing every mention of the common word that is determined to be the person's name is advantageous to ensure that patient privacy is preserved. The disclosed techniques are therefore concerned with resolving or otherwise avoiding false negatives rather than false positives.

Accordingly, the computer system 106 can remove the identified instances of the PHI from the EHR in step E. In the example described above, the computer system 106 can remove all instances of “Smithkowski” that appear in the EHR. This includes removing “Smithkowski” from the in context locations in the EHR as well as in the out of context locations in the EHR. The resulting EHR data may no longer personally identify the patient by their name “Smithkowski.”

The computer system 106 can then transmit the cleaned EHR to the user device 104 in step F. The computer system 106 can also store the cleaned EHR in a data store to be retrieved and/or used at a future time. All instances of PHI can be removed from the cleaned EHR. The resulting cleaned EHR can then be used, by the clinician at the user device 104 and/or by one or more other computing systems and/or users, to perform some action. For example, the cleaned EHR can be used to diagnose and treat the patient associated with the EHR. The cleaned EHR can also be used to perform medical-related research, studies, and/or analysis. The cleaned EHR data can also be used to search conditions or other medical information across large groups of patient records without compromising patient privacy. Thus, the cleaned EHR can be used to improve diagnosis, treatment, and research in the medical industry while meeting EHR compliance requirements and preserving patient privacy.

FIG. 1B is a block diagram of an NLP deep learning model pipeline 150 used for performing the disclosed techniques. The computer system 106 (e.g., refer to FIG. 1A) can use deep learning modeling that can be trained on various datasets to accurately identify instances of PHI and remove such instances from EHR data. The pipeline 150 can include a tokenizer 152, a tagger 154, a parser 156, and a named entity recognizer 158. Text in EHR data can be run through the pipeline 150 in order to train the deep learning model to identify and extract all instances of PHI in the EHR data.

The pipeline 150 can be pre-trained on common text corpus (e.g., such as with blogs, news, comments, and other text sources). The pipeline 150 can also be fine-tuned on medical specific text corpus (e.g., such as datasets). The deep learning model can be trained to pick up on a variety of signals, including but not limited to part of speech and vector embeddings of surrounding words. A performance metrics score threshold can also be adjusted to a desired level based on accuracy, recall, and/or one or more other factors. For example, the pipeline 150 can be trained and refined to achieve best recall.

The deep learning model pipeline 150 can accurately identify words and phrases that appear in a context. PHI that is detected from the pipeline 150 can be used to generate a dictionary. The dictionary, as described throughout this disclosure, can then be applied, by the computer system 106, to other parts of the EHR data in order to identify phrases or words that do not appear in context. Since the dictionary can be built using the pipeline 150, a confidence score can be 1 on a scale where 1 is a highest level of confidence.

Moreover, rule-based models can be generated and used to extract other information from EHR data. Rule-based models can be trained to extract prefixes and metadata from EHR data. Such models can be trained using manual extraction of prefixes and metadata from training medical records datasets. As a result, confidence scores for such rule-based models can also be 1 on a scale where 1 is a highest level of confidence.

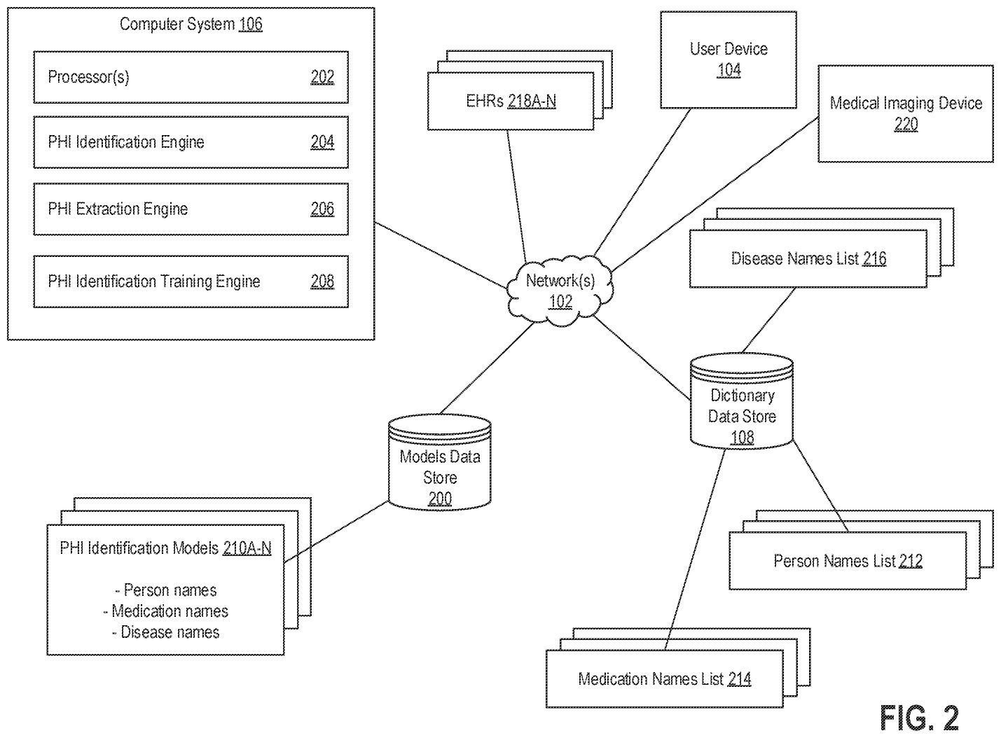

During runtime, the computer system 106 can therefore use the disclosed techniques (e.g., the pipeline 150 and the rule-based models) in order to accurately remove all instances of PHI from EHR data, thereby preserving patient privacy. FIG. 2 illustrates some components of the computing landscape of FIG. 1A that can be used to perform the techniques described herein. The user device 104, computer system 106, medical imaging device 220, dictionary data store 108, and models data store 200 can communicate (e.g., wired and/or wireless) via the network(s) 102. Although depicted as separate components, one or more of the components described herein can be part of a same computing system, network of computing devices and/or servers, and/or devices. For example, the computer system 106 can be part of a same system as the user device 104. As another example, the models data store 200 and the dictionary data store 108 can be part of a same data store and/or data warehouse or other form of storage (e.g., cloud storage, storage at the computer system 106, etc.). As yet another example, the medical imaging device 220 can be part of the computer system 106 and/or the user device 104.

The user device 104, as described in reference to FIG. 1A, can be any computing device, mobile device, laptop, computer, tablet, etc. that can be used by a clinician or other practitioner in a medical setting. The user device 104 can include input and output devices for displaying information to the clinician and receiving user input.

The medical imaging device 220 can be any device that is used in a medical setting to capture images of a patient and/or portions of a patient's body. The medical imaging device 220 can, for example, be configured to capture mammograms, x-rays, brain images, CT scans, MRI scans, etc. In some implementations, the medical imaging device 220 can also include the user device 104 and/or another type of user interface display for presenting information and receiving input from the clinician.

As described throughout this disclosure, the computer system 106 can be configured to identify instances of PHI in EHR data and extract or otherwise remove the PHI from the EHR data. The computer system 106 can also be configured to update dictionaries of PHI with new PHI phrases and/or a PHI word. Accordingly, the computer system 106 can include processor(s) 202, a PHI identification engine 204, a PHI extraction engine 206, and a PHI identification training engine 208. Although not depicted, the computer system 106 can also include a communication interface for providing communication between one or more of the components described herein.

The processor(s) 202 can be configured to perform one or more of the operations described herein.

The PHI identification engine 204 can be configured to identify instances of PHI in EHR data using one or more PHI identification models 210A-N. The PHI identification models 210A-N can be retrieved from the models data store 200 by the PHI identification engine 204. The models 210A-N can be trained to identify instances of PHI in both in context and out of context locations in different types of EHRs 218A-N. The models 210A-N can be deep learning models with keras, main layer: bi-directional LSTM+CRF (Conditional Random Field). The models 210A-N can be one or more other deep learning models such as convolutional neural networks (CNNs).

For example, one or more of the models 210A-N can be trained to identify person names that appear in known contexts in the EHRs 218A-N. One or more of the known contexts can include headers, footers, titles, contextual words before, and contextual words after. In some implementations, headers, footers, and/or titles may not be known contexts, as described in some scenarios throughout this disclosure. Known contexts can also include, but are not limited to, text fields that are designated for receiving patient names and signature blocks/lines. Sometimes, one or more of the models 210A-N may identify names that appear in known contexts and names that are already identified in a dictionary. Relying only on names that appear in a generic dictionary may return some quantity of false positives. This is the case because a generic dictionary can contain many words and phrases that are not considered names. Using a dictionary containing words and phrases that are considered names can be advantageous because fewer or no false positives may be returned.

The models 210A-N can then be trained to build a dictionary (or in some implementations, add to an existing dictionary) with recognized person names or other PHI and use the dictionary to filter unrecognized names or PHI from other parts of the EHRs 218A-N. Sometimes, the models 210A-N may identify a token (e.g., word, phrase, letter(s), etc.) that is potentially part of a person's name. In such scenarios, the models 210A-N can be trained to look at next tokens to check whether such tokens should be included as part of the person's name and therefore removed from the EHRs 218A-N. The models 210A-N can be trained, for example, to identify “,” and/or “−,” which can indicate that the token(s) should be considered part of the person's name.

The following exemplary code can be implemented to perform one or more of the techniques described herein. Similar code can also be used to implement the techniques described herein.

def analyze(self, text, entities, nlp_artifacts):
results = [ ]
# Match by regex
for entity in entities:
if entity not in self.supported_entities:
continue
if entity == “PERSON”:
results.extend(
self.match_by_regex(text, self.REGEX_CONTEXT_WORDS_BEFORE,
self.DEFAULT_EXPLANATION, 1))
results.extend(
self.match_by_regex(text, self.REGEX_CONTEXT_WORDS_AFTER,
self.DEFAULT_EXPLANATION, 1))
results.extend(
self.match_by_regex(text, self.REGEX_NAME_AT_FOOTER,
self.FOOTER_EXPLANATION))
# Matching by dictionary can be too aggressive, too many false positives, so can
temporarily disable
# Match by dictionary
# doc = nlp_artifacts.doc
# nip = nlp_artifacts.nlp_engine.nlp[self.supported_language]
# person name matcher = Matcher(nlp.vocab)
# person_name_matcher.add(“person_names”, self.person_name_patterns)
# dictionary_matches = person_name_matcher(doc)
# for match_id, start, end in dictionary_matches:
# explanation = self_build_spacy_explanation(self.__class__.__name__,
self.ner_strength, self.DICTIONARY_MATCH_EXPLANATION)
# span = doc[start:end]
# result = RecognizerResult(self.ENTITIES[0], span.start_char, span.end_char,
self.ner_strength, explanation )
# results.append(result)
# Now build a dictionary from recognized person names and use it to filter
unrecognized names from other parts of the text
person_names = [ ]
doc = nlp_artifacts.doc
nip = nlp_artifacts.nlp_engine.nlp[self.supported_language]
for e in nlp_artifacts.entities:
if e.label_ != “PERSON”:
continue
for token in e:
if token.is_alpha:
person_names.append(token)
for r in results:
span = doc.char_span(r.start, r.end)
if span is None:
continue
for token in span:
if token.is_alpha and not self.token_included(person_names, token):
person_names.append(token)
patterns = [nlp(n.text) for n in person_names]
name_matcher = PhraseMatcher(nlp.vocab, attr=‘LOWER’)
name_matcher.add(“name_patterns”, patterns)
matches = name_matcher(doc)
for _, start, _ in matches:
# if self.token_included(person_names, doc[start]):
# continue
# When we found a token that is potentially part of person name, we move
forward to the next tokens to check whether we should also include them as part
of the name
next_idx = doc [ start].i
while True:
next_idx += 1
if next_idx >= len(doc):
break
token = doc[next_idx]
if self.token_included(person_names, token):
break
if token.text == ‘,’ or token.text == ‘-’ or token. shape_.startswith(“X”):
continue
else:
break
span = doc [start:next_idx]
explanation = self.build_spacy_explanation(self.__class__.__name__,
self.ner_strength, self.DICTIONARY_MATCH_EXPLANATION)
result = RecognizerResult(self.ENTITIES[0], span.start_char, span.end_char,
self.ner_strength, explanation)
results, append(result)
return results

Below is example code for a config file consisting of hyperparameters for a transformer-based model. Similar code can be used to perform the techniques described herein and to train one or more of the models 210A-N described throughout this disclosure.

[paths]
train = “/data/training”
dev = “/data/testing”
vectors = null
init_tok2vec = null
[system]
gpuallocator = “pytorch”
seed = 0
[nip]
lang = “en”
pipeline = [“transformer”, “ner”]
tokenizer = {“@tokenizers”:“spacy.Tokenizer.v1”}
batchsize =128
disabled = [ ]
before_creation = null
after_creation = null
after_pipeline_creation = null
[components]
[components.ner]
factory = “ner”
moves = null
update_with_oracle_cut_size = 100
[components.ner.model]
architectures = “spacy.TransitionBasedParser.v2”
state_type = “ner”
extra_state_tokens = false
hidden_width = 64
maxout_pieces = 2
use_upper = false
nO = null
[components.ner.model.tok2vec]
architectures = “spacy-transformers.TransformerListener.v1”
grad_factor =1.0
pooling = {“@layers”:“reduce_mean.v1”}
upstream = “*”
[components.transformer]
factory = “transformer”
max_batch_items = 4096
set_extra_annotations = {“@annotation_setters”:“spacy-
transformers.null_annotation_setter.v1”}
[components.transformer.model]
architectures = “spacy-transformers.TransformerModel.v1”
name = “roberta-base”
[components.transformer.model.getspans]
@span_getters = “spacy-transformers.strided_spans.v1”
window =128
stride = 96
[components.transformer.model.tokenizer_config]
use_fast = true
[corpora]
[corpora.dev]
@readers = “spacy.Corpus.v1”
path = ${ paths.dev}
max_length = 0
gold_preproc = false
limit = 0
augmenter = null
[corpora.train]
@readers = “spacy.Corpus.v1”
path = ${ paths.train}
max_length = 500
gold_preproc = false
limit = 0
augmenter = null
[training]
accumulate_gradient = 3
dev_corpus = “corpora.dev”
train_corpus = “corpora.train”
seed = ${system.seed}
gpu_allocator = ${system.gpu_allocator}
dropout = 0.1
patience = 1600
max_epochs = 0
max_steps = 20000
eval_frequency = 200
frozen_components = [ ]
before_to_disk = null
[training.batcher]
@batchers = “spacy.batch_by_padded.v1”
discard_oversize = true
size = 2000
buffer = 256
get_length = null
[training.logger]
@loggers = “spacy.ConsoleLogger.v1”
progress_bar = false
[training.optimizer]
optimizers = “Adam.v1”
beta1 = 0.9
beta2 = 0.999
L2_is_weight_decay = true
L2 = 0.01
gradclip =1.0
use_averages = false
eps = 0.00000001
[training.optimizer.learnrate]
@schedules = “warmup_linear.v1”
warmup_steps = 250
total_steps = 20000
initial_rate = 0.00005
[training.scoreweights]
ents_per_type = null
ents_f = 1.0
ents_p = 0.0
ents_r = 0.0
[pretraining]
[initialize]
vectors = null
init_tok2vec = ${paths.init_tok2vec}
vocab_data = null
lookups = null
before_init = null
after_init = null
[initialize.components]
[initialize.tokenizer]

The models 210A-N can be trained using NLP techniques to identify instances of PHI that appear in context in EHR data. The models 210A-N can also receive text as input where the text is derived using optical character recognition techniques. The models 210A-N can then be trained and improved to identify the same PHI in out-of-context locations in the EHR data. Thus, the models 210A-N can be trained to identify instances of PHI as the PHI appears in text fields, text boxes, signature blocks, margins, headers, footers, and/or images. The models 210A-N can also identify instances of PHI as the PHI appears in with contextual clues such as prefixes (e.g., Mr., Mrs., Miss, Ms., Dr., Nurse, Jr., and Sr.)., operators, and/or delimiters.

During runtime, the PHI identification engine 204 can receive EHRs 218A-N from the user device 104, the medical imaging device 220, and/or a data store for storing the EHRs 218A-N. Using the PHI identification models 210A-N, the PHI identification engine 204 can identify instances of PHI that appear anywhere in the received EHRs 218A-N, including conventional (e.g., typical) locations or contexts and unconventional (e.g., atypical) locations or contexts.

Once the PHI identification engine 204 identifies the PHI in context within the EHRs 218A-N, the PHI identification engine 204 can add the PHI to one or more dictionaries stored in the dictionary data store 108, if the PHI does not already exist in the one or more dictionaries. As described throughout, the dictionary data store 108 can store different lists of words and/or phrases that have been and are identified as PHI. As examples, the dictionary data store 108 can store person names list 212, medication names list 214, and/or disease names list 216. When new PHI are identified by the PHI identification engine 204, the new PHI can be added to the corresponding list (e.g., dictionary) that is stored in the dictionary data store 108. As a result, the lists 212-216 can be expanded to include different types of PHI. These lists 212-216 can then be used by the computer system 106 to more accurately identify and extract PHI in subsequent EHRs 218A-N. Sometimes, one list (e.g., dictionary) can be used for identifying different types of PHI, including but not limited to person names, medication names, and disease names.

As mentioned above, the PHI identification engine 204 can identify all instances of the PHI that appear out of context in the EHRs 218A-N. Out of context locations can include locations in the EHRs 218A-N, such as margins, headers, footers, non-designated input fields, and/or other portions of the EHRs 218A-N. Out of context locations can also include instances of the PHI where the PHI is not surrounded by contextual clues, such as prefixes, operators, and/or delimiters. The PHI identification engine 204 can use the updated lists 212-216 to locate PHI in the out of context locations and label those as instances of the PHI. Once all instances of the PHI are labeled in the EHRs 218A-N, those instances can be removed.

The PHI extraction engine 206 can be configured to extract or remove any labeled instances of PHI that appear in the EHRs 218A-N. For example, the PHI extraction engine 206 can receive the EHRs 218A-N from the PHI identification engine 204 after the PHI instances have been labeled and tagged as PHI. The PHI extraction engine 206 can then remove all labeled instances of the PHI from the EHRs 218A-N. Removing the labeled instances of the PHI can include blacking or whiting out the PHI. Removing the PHI can also include any other means of making the PHI unreadable and undecipherable. For example, removing the PHI can include replacing the PHI with random strings of text, numbers, and/or symbols.

By removing all instances of the PHI from the EHRs 218A-N, the PHI extraction engine 206 can produce a cleaned version of the EHRs 218A-N. This cleaned version of the EHRs 218A-N can then be used by a clinician or other practitioner at the user device 104 to perform one or more actions, as described further below. The cleaned version of the EHRs 218A-N can also be transmitted over different networks to one or more computing systems and/or devices that are remote or separate from an in-hospital or other user infrastructure. Because the EHRs 218A-N no longer include readable, decipherable PHI, the EHRs 218A-N can be transmitted and used by other relevant stakeholders without jeopardizing patient privacy.

The PHI identification training engine 208 can be configured to train one or more of the PHI identification models 210A-N to identify instances of PHI from EHRs 218A-N. One or more machine learning techniques, such as deep neural (DL) networks and/or convolutional neural networks (CNNs) can be used for training purposes. The PHI identification training engine 208 can train one or more person names models, medication names models, and/or disease names models to use the lists 212-216 in identifying instances of PHI throughout EHRs 218A-N. For example, the PHI identification models 210A-N can be trained to identify instances of PHI using contextual clues, such as prefixes, delimiters, and/or operators. Optical character recognition techniques and/or NLP techniques can also be used to train the PHI identification models 210A-N to identify known PHI from the lists 212-216 in the EHRs 218A-N, regardless of where the PHI appears in the EHRs 218A-N.

For example, the PHI identification models 210A-N can be trained to identify PHI in out of context locations by using EHR training data where person, medication, and/or disease names are identified and annotated as PHI in headers and footers. The PHI identification models 210A-N can also be trained to identify PHI in out of context locations by using EHR training data where person, medication, and/or disease names are identified and annotated as PHI when preceded by prefixes (e.g., Mr., Mrs., Doctor, dosage amount, etc.) and when the same person, medication, and/or disease names are identified and annotated as PHI when not preceded by prefixes. Therefore, the PHI identification models 210A-N can be trained to accurately identify instances of PHI regardless of where those instances appear in the EHRs 218A-N.

Moreover, the PHI identification training engine 208 can continuously train or otherwise improve the PHI identification models 210A-N based on runtime application of the techniques described herein. For example, as the lists 212-216 are updated by the PHI identification engine 204, the PHI identification training engine 208 can train the PHI identification models 210A-N to identify the PHI that has been added to the lists 212-216. Continuous improvements of the models used herein can be advantageous to improve the computer system 106's ability to accurately remove all PHI from EHRs. As a result, compliance requirements can be met and patient privacy can be preserved, regardless of where or how PHI appears in EHR data.

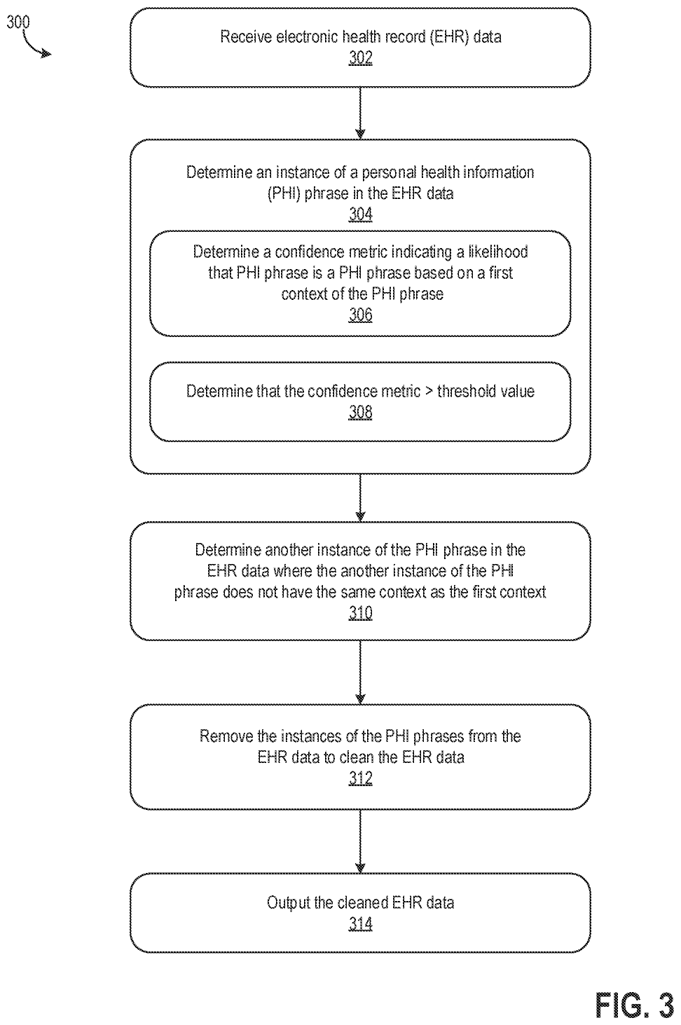

FIG. 3 is a flowchart of a process 300 for removing PHI from EHR data. The process 300 can be performed by the computer system 106 described herein (e.g., refer to FIG. 1A). The process 300 can also be performed by one or more other computing systems, networks of computers, servers, cloud services, computers and/or devices. For illustrative purposes, the process 300 is described from a perspective of a computer system.

Referring to the process 300, the computer system can receive EHR data in 302. As described throughout this document, the EHR data can be received from user devices of clinicians or other medical professionals (e.g., the user device 104 in FIG. 1A), medical imaging devices (e.g., the medical imaging device 220 in FIG. 2), and/or data stores that securely maintain copies of the EHR data (e.g., a data store of an in-hospital infrastructure). The EHR data can include documentation with information about a patient, their medical history, and their personal information. The EHR data can also include image data regarding medical conditions of the patient. The image data can have personal information or other identifying information about the patient.

In 304, the computer system can determine an instance of a PHI phrase in the EHR data. The computer system can use one or more machine learning models having NLP techniques to determine an instance of a PHI phrase. The models can be trained to identify phrases in the EHR data and assign confidence values to the phrases. The confidence values (e.g., a confidence metric) can indicate a likelihood that an identified phrase is a PHI phrase. The confidence values can be assigned based on a context that the phrase is identified in. In other words, the confidence metric can be based on a first context of the PHI phrase (306). The computer system can determine that the confidence metric is greater than a threshold value (308). The higher the confidence metric, the more likely that the identified instance of the phrase is a PHI phrase. The threshold value can therefore indicate a level of confidence needed to determine that the phrase is a PHI phrase that can and should be removed from the EHR data.

Using the techniques described herein, the computer system can detect phrases, which can include words, symbols, and/or punctuation, that likely represent a person's name, medication name, disease name, or other personally identifying information. Sometimes, the models that are used to identify PHI phrases can utilize optical character recognition (OCR) techniques to identify phrases or words in the EHR data. OCR techniques can be used where the EHR data does not appear in a text format. For example, the EHR data can be a scanned document, a PDF, image data, video data, or other non-text formatted documents, information, or data.

Still referring to 304, the computer system can use the models to identify particular phrases that have been defined as PHI phrases. For example, the PHI phrases can be defined in one or more dictionaries, which can be accessed and used by the computer system to identify PHI phrases in the EHR data. The computer system can also identify phrases as PHI phrases based on contextual clues, such as the first context mentioned above. The first context can include instances of phrases or a word that follow a prefix, such as Doctor, Nurse, Dr., Mr., Miss, Ms., or Mrs. One or more example prefixes include regular expression patterns, including but not limited to “Signed by,” “signed by,” “Signed electronically by,” “signed electronically by,” “Electronically signed by,” “electronically signed by,” “Dear,” “dear,” “Name,” “name,” “NAME,” “Patient,” “patient,” “PATIENT,” and/or “RE:.” Phrases following such prefixes can be identified as PHI phrases (304), assigned high confidence metrics (306), and determined to exceed the threshold value for the confidence metrics (308).

Example code that can be implemented to identify the prefixes is included below. Similar code can also be implemented to identify the same, similar, and/or additional prefixes in EHR data.
r″(?:[Sls]igned by)“,
r″(?:[Sls]igned electronically by)“,
r″(?:electronically signed by)“,
r″(?:[Dld]ear)“,
r″(?:[Nn][Aa][Mm][Ee]:)“,
r″(?:[Pp][Aa][Tt][Ii][Ee][Nn][Tt]:)“,
r″(?:RE:)“,
r″(?:(?:Dr1Mr1Ms1Mrs)[\.]*)”

Similarly, the first context can include instances of phrases or a word that appear in a particular location in the EHR data where PHI phrases typically appear. The first context can include a text field, input field, data field, or other location in the EHR data that is intended to receive PHI phrases and/or is where PHI phrases are typically found. For example, if the EHR data contains a line at the top of the EHR data for clinicians to input patient names, then any name that appears on that line can be identified as a PHI phrase with a high confidence metric.

The confidence metric can be a numeric, Boolean, or string value. The confidence metric can be assigned on a numeric scale, such as a value between −1 and 1, 0 and 1, 1 and 10, 1 and 100, etc. Sometimes, each PHI phrase can be assigned predefined, fixed values instead of confidence metrics. Any phrase or word having one of the predefined, fixed values is categorized as a PHI phrase that can be removed from the EHR data. As an example, all identified person names can be assigned a value of 1, all identified disease names can be assigned a value of 2, and all identified medication names can be assigned a value of 3. Accordingly, any phrases or words having values of 1, 2, or 3 can be removed from the EHR data.

If the PHI phrase is defined in a dictionary, as another example, the PHI phrase can be assigned a higher confidence metric than a phrase that does not appear in the dictionary. A higher confidence metric can be 0.9 or a 1.0 on a scale where 1.0 is a highest value that can be assigned. If, on the other hand, a PHI phrase is identified in the EHR data because it follows a prefix such as Doctor or Nurse but the PHI phrase does not appear in the dictionary, then the PHI phrase can be assigned a lower confidence metric, such as a 0.1 on a scale, where 0 is a lowest value that can be assigned. Other assignments of confidence metrics are also possible.

Next, the computer system can determine another instance of the PHI phrase in the EHR data where the another instance of the PHI phrase does not have the same context as the first context (310). In other words, since the computer system has identified the PHI phrase, the computer system can now search, using the one or more machine learning models, the EHR data for that patient/subject for any other instances where the PHI phrase appears. It is possible that the PHI phrase may not appear in the same context as the first instance (e.g., the first context) of the PHI phrase that was identified in 304. For example, the first context can include a prefix. A second context where the PHI phrase is identified can be a footer of an EHR document. The second context can also be the same PHI phrase from the first context but without the prefix. The same PHI phrase can therefore appear in other locations in the EHR data. The same PHI phrase can also appear with same or different contextual clues as the PHI phrase in the first context.

As described throughout this disclosure, the PHI phrase can appear in different contexts. Sometimes, the context of the another instance of the PHI phrase may be such that a confidence metric for that another instance is not above the threshold. For example, the another instance can be assigned a confidence metric that it is a PHI phrase based only on a single, local context. Thus, the confidence metric can be lower than a specified threshold. The another instance can also be assigned a confidence metric that is based on the another instance being out of context or in a limited context, but this confidence metric can be above a threshold required for extraction or obfuscation since the PHI phrase had already been identified earlier in the first context. The another instance of the PHI phrase can be extracted in the latter case because it has a confidence metric that exceeds the threshold level. . For example, the PHI phrase can appear in locations in the EHR data that do not typically have the PHI phrase or the PHI phrase can simply appear out of context. The PHI phrase may also appear without any of the prefixes described above. The PHI phrase can appear in a header or footer of a PDF EHR document or other type of EHR data. The PHI phrase can also appear handwritten in a corner or margin of the EHR data. The PHI phrase may appear in a block of text in a body of the EHR data without any contextual clues such as the prefixes described above. The PHI phrase can also overlay EHR image data. Moreover, the PHI phrase can appear in a context that includes one or more literals, delimiters, and/or operators that are different than the first context of the PHI phrase. Thus, when the another instance of the PHI phrase appears out of context, or otherwise in a context that is not the same as the first context, the another instance of the PHI phrase can be assigned a lower confidence metric than the instance of the PHI phrase in the first context. Regardless of the lower confidence metric based on the local context along, the another instance of the PHI phrase can be identified and tagged as PHI that can be removed from the EHR data. As used above, the term local means with a few, e.g., 3 words of the content in question (e.g., the PHI content candidate) or metadata that applies directly to the content in question.

The computer system can then remove the instances of the PHI phrases from the EHR data to clean the EHR data (312). The computer system can remove all of the PHI phrases that have been identified in the EHR data. Removing the PHI phrases can include blacking or whiting them out in the EHR data. Removing the PHI phrases can also include making the PHI phrases undecipherable or otherwise unreadable, as described above. Removing the PHI phrases can also include replacing the PHI phrases with random strings of numbers, letters, and/or characters. As a result of removing the PHI phrases, patient privacy associated with the EHR data can be preserved. Personal information of the patient may not be gleaned from the cleaned EHR data. The disclosed process 300 is beneficial because it can provide for removing all instances of the PHI phrases, regardless of where such PHI phrases appear in the EHR data.

The computer system can then output the cleaned EHR data in 314. Outputting the cleaned EHR data can include storing the cleaned EHR data in a data store for future use and/or retrieval. For example, the cleaned EHR data can be used in medical research. Outputting the cleaned EHR data can also include transmitting the cleaned EHR data back to the user device. Sometimes, outputting the cleaned EHR data can include providing the cleaned EHR data directly to the patient associated with the EHR data.

Outputting the cleaned EHR data can also include transmitting the cleaned EHR data to another computing device that can be used by clinicians, patients, researchers, and/or other medical professionals to determine treatment, diagnosis, research, or other information about the patient's medical condition. The cleaned EHR data can be used to generate recommendations for types and/or aspects of surgeries or other procedures for the particular patient. For example, cleaned EHR data can be used to recommend transcranial magnetic stimulation, pharmaceutical and drug treatment, counseling, and/or lifestyle changes. Moreover, the cleaned EHR data can be used for continuous tracking of conditions of the particular patient and/or a cohort of similar patients. One or more purposes of the cleaned EHR data are also possible since the cleaned EHR data does not have any personally identifying information that would compromise patient privacy.

As an illustrative example, the cleaned EHR data can be brain imaging data of a patient. The cleaned EHR data can then undergo backend analysis such as image processing such that additional images of the brain can be produced. This backend analysis can be performed on the cleaned EHR data to generate additional data and/or information about the patient's brain without exposing personal information or jeopardizing the patient's privacy.

Sometimes, outputting the cleaned EHR data can include using the cleaned EHR data as input to continuously improve the computer system (e.g., refer to FIG. 2). For example, if a PHI phrase is identified in a first, known context (e.g., following a prefix) and another instance of the same PHI phrase is identified in a second, different context (e.g., in a footer of the EHR data), then the machine learning models used by the computer system can be trained to learn to identify the PHI phrase in the second context as well as the first context. Using such models, the computer system can more accurately detect and extract PHI phrases in subsequent EHR data, regardless of where such PHI phrases appear in the EHR data.

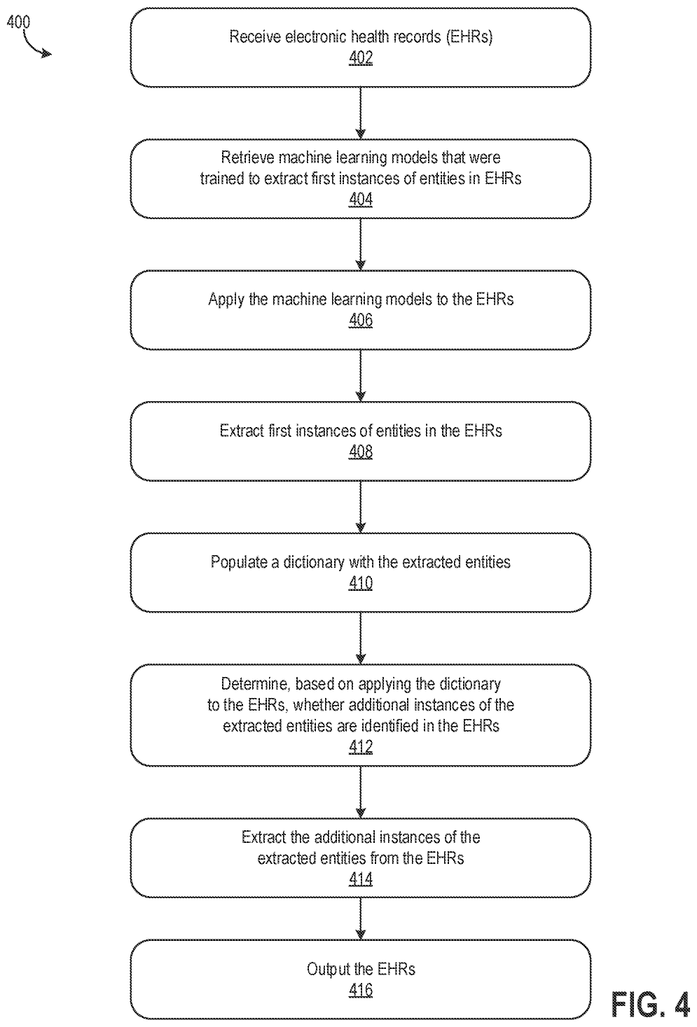

FIG. 4 is a flowchart of a process 400 for building a dictionary that can be used to identify and remove PHI from EHR data. The dictionary can be used to identify PHI in different contexts and extract the PHI from EHR data. Typically, names or other PHI that appear out of context, such as in a header of the EHR data, may go unrecognized by existing NLP systems. Such NLP systems may not register the out of context locations as relevant when identifying and extracting PHI phrases, but may register in context instances (e.g., “Mr. Hans”) of the PHI phrases as relevant. The process 400 can be advantageous to identify PHI phrases that appear out of context, especially since oftentimes, PHI phrases may not appear in certain fields, locations, or contexts across different types of EHR data (e.g., scanned documents, image data, editable documents, etc.).

The process 400 can be performed by the computer system 106 described herein (e.g., refer to FIG. 1A). The process 400 can also be performed by one or more other computing systems, networks of computers, servers, cloud services, computers and/or devices. For illustrative purposes, the process 400 is described from a perspective of a computer system.

Referring to the process 400, the computer system can receive EHRs in 402. Refer to block 302 in FIG. 3 for further discussion.

In 404, the computer system can retrieve one or more machine learning models that were trained to extract first instances of entities in EHRs. The models can be trained to extract first instances of entities in predefined textual locations of the EHRs. The models can be trained using one or more rules to identify entities in different predefined textual locations. For example, one rule can indicate that a noun phrase following a prefix such as “Mr.” or “Mrs.” likely is a person's name and thus an entity that can be extracted from the EHRs. Another rule can indicate that a noun phrase appearing in certain text or input fields (e.g., a text field having a prefix of “Patient Name”) likely is a person's name and thus an entity that can be extracted from the EHRs.

One or more other rules can identify patterns that may appear in different PHI phrases. For example, some person's names can be hyphenated. Thus, a rule can indicate that a noun phrase appearing with a hyphen can likely be a person's name and thus an entity that can be extracted from the EHRs. As another example, a rule can indicate that a noun phrase ending with one of a plurality of identified suffixes can likely be a drug or medication name and thus an entity that can be extracted from the EHRs. For example, the suffixes can include -dipine, -ine, -sone, -statin, -vir, and -zide. One or more other suffixes that are common for drug or medication names can also be included. Another rule can indicate that a noun phrase ending with one of a plurality of identified suffixes can likely be a disease name and thus an entity that can be extracted. For example, these suffixes can include -algia, -emia, -itis, -lapse, and -lepsy. One or more other suffixes that are common for diseases can also be included. Other rules are possible. Rules can be generated and/or updated incrementally as more or different instances of PHI phrases are identified, extracted, or otherwise identified in EHR data.

The first instances can include different contexts where entities (e.g., PHI phrases or word(s)) are likely to be found in the EHRs. For example, as described in reference to block 304 in FIG. 3, the first instances of the entities can include first contexts. The entities can be person's names, medication names, disease names, and other personally identifying information. The entities can be stored in a dictionary (e.g., refer to the dictionary data store 108 in FIG. 2). The computer system can use the dictionary and the machine learning models to identify where entities that are defined by the dictionary appear in the EHRs. A person's name can appear with a prefix in a text field and/or input field in an EHR document. This can be the first instance of the entity since the person's name is appearing in a known context (e.g., with a prefix, in a text and/or input field). The one or more machine learning models can be trained to identify such first instances of entities.

The computer system can then apply the machine learning models to the EHRs in 406. The computer system can accordingly extract first instances of the entities in the EHRs (408). As mentioned above, once the machine learning models identify the first instances of the entities, such instances can be removed from the EHRs. For example, if the computer system, using the machine learning models, identifies a person's name in a text field within a body of an EHR document, the computer system can subsequently remove the person's name from the text field.

The computer system can also populate a dictionary with the extracted entities in 410. The computer system, based on applying the one or more machine learning models to the EHRs, can identify entities that appear in the first instances (e.g., first contexts) indicative of PHI phrases or words. However, the identified entities may not be known phrases or words in the dictionary. Thus, the identified entities can be added to the dictionary so that the computer system can more accurately identify the same entities in other types and/or forms of EHRs. The identified entities can be labeled in the document and stored with the labels in a database (e.g., refer to the dictionary data store 108). For example, the computer system can identify an entity “Goldensmith-Child” because this entity can appear in a first, known instance in which “Mrs.” prefix precedes “Goldensmith-Child.” “Goldensmith-Child” may not be a known word or phrase, and therefore may not appear in the dictionary. However, because “Goldensmith-Child” follows a known prefix, “Goldensmith-Child” is likely to be a PHI phrase (e.g., a person's name). Accordingly, the computer system can add “Goldensmith-Child” to the dictionary. When the computer system analyzes other types and/or forms of EHRs (or the same EHR where “Goldensmith-Child” was identified), the computer system can more accurately and quickly identify and extract other instances of “Goldensmith-Child.” If the extracted entities are already defined in the dictionary, then they may not be added to the dictionary. The computer system can skip block 410.

The computer system can determine, based on applying the dictionary to the EHRs, whether additional instances of the extracted entities are identified in the EHRs (412). In other words, the computer system can determine whether the extracted entities appear in other locations in the EHRs. In the example above, the computer system can perform a second pass through the EHR document where the name “Goldensmith-Child” was identified in a first instance and extracted. During the second pass through, the computer system can search for other instances of “Goldensmith-Child” that may not be the first instance. Thus, the other instances of “Goldensmith-Child” can be out of context or in locations in the EHRs where PHI phrases likely do not appear. The computer system can, for example, identify another instance of “Goldensmith-Child” in a header or footer of the EHR document. The computer system can also identify another instance of “Goldensmith-Child” in a margin, handwritten, in a signature block, in a block of text, without prefixes, or otherwise not in a known context, such as the first contexts described in reference to FIG. 3.

The computer system can extract the additional instances of the extracted entities from the EHRs in 414. In the example above, the computer system can extract “Goldensmith-Child” from the header, footer, margin, handwriting, signature block, block of text, or other unknown context. The computer system can therefore increase the likelihood that all instances of the extracted entities are removed from the EHRs to produce cleaned EHRs.

The computer system can also output the EHRs in 416. As described in reference to block 314 in FIG. 3, the computer system can output cleaned EHRs, which can be used for subsequent analysis, post-processing, research, diagnosis, and/or treatment of patients in a medical setting.

The process 400 is described from a perspective of identifying and extracting entities from a plurality of EHRs. In some implementations, the computer system can implement the process 400 as a single lane pipeline. EHRs can be analyzed and cleaned using the process 400 all at once. Sometimes, the EHRs can be received from one medical institution and processed in batch. Sometimes, the EHRs can be received from multiple different medical institutions and processed in batch. In yet some implementations, the EHRs can be received from one or more medical institutions at different times and processed using the process 400 at different times.

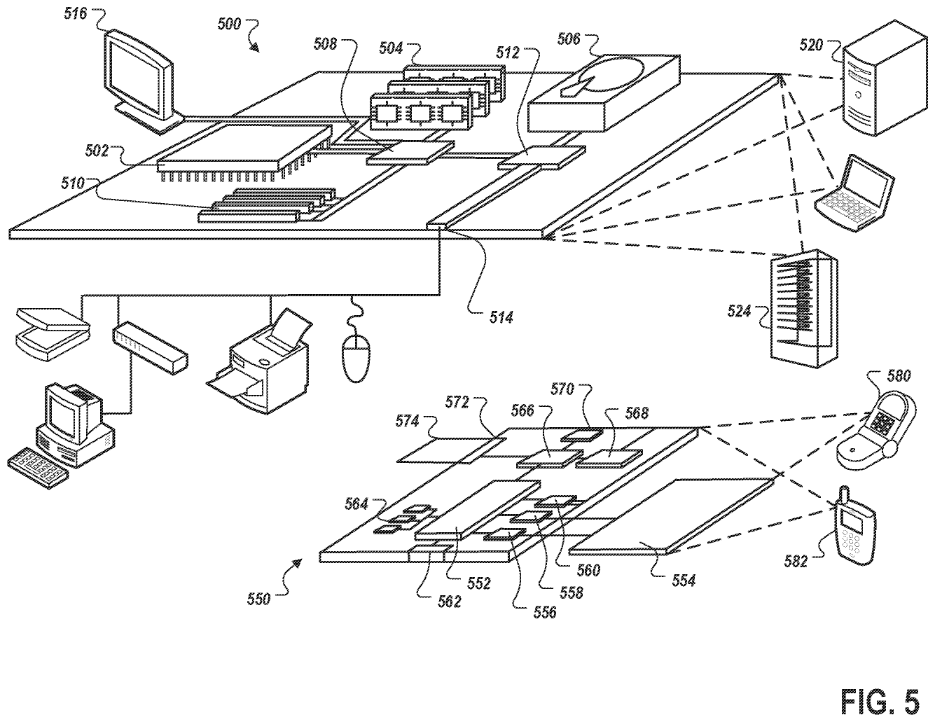

FIG. 5 shows an example of a computing device 500 and an example of a mobile computing device that can be used to implement the techniques described here. The computing device 500 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, and other similar computing devices. The components shown here, their connections and relationships, and their functions, are meant to be exemplary only, and are not meant to limit implementations of the inventions described and/or claimed in this document.

The computing device 500 includes a processor 502, a memory 504, a storage device 506, a high-speed interface 508 connecting to the memory 504 and multiple high-speed expansion ports 510, and a low-speed interface 512 connecting to a low-speed expansion port 514 and the storage device 506. Each of the processor 502, the memory 504, the storage device 506, the high-speed interface 508, the high-speed expansion ports 510, and the low-speed interface 512, are interconnected using various busses, and can be mounted on a common motherboard or in other manners as appropriate. The processor 502 can process instructions for execution within the computing device 500, including instructions stored in the memory 504 or on the storage device 506 to display graphical information for a GUI on an external input/output device, such as a display 516 coupled to the high-speed interface 508. In other implementations, multiple processors and/or multiple buses can be used, as appropriate, along with multiple memories and types of memory. Also, multiple computing devices can be connected, with each device providing portions of the necessary operations (e.g., as a server bank, a group of blade servers, or a multi-processor system).

The memory 504 stores information within the computing device 500. In some implementations, the memory 504 is a volatile memory unit or units. In some implementations, the memory 504 is a non-volatile memory unit or units. The memory 504 can also be another form of computer-readable medium, such as a magnetic or optical disk.

The storage device 506 is capable of providing mass storage for the computing device 500. In some implementations, the storage device 506 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The computer program product can also be tangibly embodied in a computer- or machine-readable medium, such as the memory 504, the storage device 506, or memory on the processor 502.

The high-speed interface 508 manages bandwidth-intensive operations for the computing device 500, while the low-speed interface 512 manages lower bandwidth-intensive operations. Such allocation of functions is exemplary only. In some implementations, the high-speed interface 508 is coupled to the memory 504, the display 516 (e.g., through a graphics processor or accelerator), and to the high-speed expansion ports 510, which can accept various expansion cards (not shown). In the implementation, the low-speed interface 512 is coupled to the storage device 506 and the low-speed expansion port 514. The low-speed expansion port 514, which can include various communication ports (e.g., USB, Bluetooth, Ethernet, wireless Ethernet) can be coupled to one or more input/output devices, such as a keyboard, a pointing device, a scanner, or a networking device such as a switch or router, e.g., through a network adapter.

The computing device 500 can be implemented in a number of different forms, as shown in the figure. For example, it can be implemented as a standard server 520, or multiple times in a group of such servers. In addition, it can be implemented in a personal computer such as a laptop computer 522. It can also be implemented as part of a rack server system 524. Alternatively, components from the computing device 500 can be combined with other components in a mobile device (not shown), such as a mobile computing device 550. Each of such devices can contain one or more of the computing device 500 and the mobile computing device 550, and an entire system can be made up of multiple computing devices communicating with each other.

The mobile computing device 550 includes a processor 552, a memory 564, an input/output device such as a display 554, a communication interface 566, and a transceiver 568, among other components. The mobile computing device 550 can also be provided with a storage device, such as a micro-drive or other device, to provide additional storage. Each of the processor 552, the memory 564, the display 554, the communication interface 566, and the transceiver 568, are interconnected using various buses, and several of the components can be mounted on a common motherboard or in other manners as appropriate

The processor 552 can execute instructions within the mobile computing device 550, including instructions stored in the memory 564. The processor 552 can be implemented as a chipset of chips that include separate and multiple analog and digital processors. The processor 552 can provide, for example, for coordination of the other components of the mobile computing device 550, such as control of user interfaces, applications run by the mobile computing device 550, and wireless communication by the mobile computing device 550.

The processor 552 can communicate with a user through a control interface 558 and a display interface 556 coupled to the display 554. The display 554 can be, for example, a TFT (Thin-Film-Transistor Liquid Crystal Display) display or an OLED (Organic Light Emitting Diode) display, or other appropriate display technology. The display interface 556 can comprise appropriate circuitry for driving the display 554 to present graphical and other information to a user. The control interface 558 can receive commands from a user and convert them for submission to the processor 552. In addition, an external interface 562 can provide communication with the processor 552, so as to enable near area communication of the mobile computing device 550 with other devices. The external interface 562 can provide, for example, for wired communication in some implementations, or for wireless communication in other implementations, and multiple interfaces can also be used.

The memory 564 stores information within the mobile computing device 550. The memory 564 can be implemented as one or more of a computer-readable medium or media, a volatile memory unit or units, or a non-volatile memory unit or units. An expansion memory 574 can also be provided and connected to the mobile computing device 550 through an expansion interface 572, which can include, for example, a SIMM (Single In Line Memory Module) card interface. The expansion memory 574 can provide extra storage space for the mobile computing device 550, or can also store applications or other information for the mobile computing device 550. Specifically, the expansion memory 574 can include instructions to carry out or supplement the processes described above, and can include secure information also. Thus, for example, the expansion memory 574 can be provide as a security module for the mobile computing device 550, and can be programmed with instructions that permit secure use of the mobile computing device 550. In addition, secure applications can be provided via the SIMM cards, along with additional information, such as placing identifying information on the SIMM card in a non-hackable manner.

The memory can include, for example, flash memory and/or NVRAM memory (non-volatile random access memory), as discussed below. In some implementations, a computer program product is tangibly embodied in an information carrier. The computer program product contains instructions that, when executed, perform one or more methods, such as those described above. The computer program product can be a computer- or machine-readable medium, such as the memory 564, the expansion memory 574, or memory on the processor 552. In some implementations, the computer program product can be received in a propagated signal, for example, over the transceiver 568 or the external interface 562.

The mobile computing device 550 can communicate wirelessly through the communication interface 566, which can include digital signal processing circuitry where necessary. The communication interface 566 can provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication can occur, for example, through the transceiver 568 using a radio-frequency. In addition, short-range communication can occur, such as using a Bluetooth, WiFi, or other such transceiver (not shown). In addition, a GPS (Global Positioning System) receiver module 570 can provide additional navigation- and location-related wireless data to the mobile computing device 550, which can be used as appropriate by applications running on the mobile computing device 550.

The mobile computing device 550 can also communicate audibly using an audio codec 560, which can receive spoken information from a user and convert it to usable digital information. The audio codec 560 can likewise generate audible sound for a user, such as through a speaker, e.g., in a handset of the mobile computing device 550. Such sound can include sound from voice telephone calls, can include recorded sound (e.g., voice messages, music files, etc.) and can also include sound generated by applications operating on the mobile computing device 550.

The mobile computing device 550 can be implemented in a number of different forms, as shown in the figure. For example, it can be implemented as a cellular telephone 580. It can also be implemented as part of a smart-phone 582, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some implementations, the computing system can be cloud based and/or centrally processing data. In such case anonymous input and output data can be stored for further analysis. In a cloud based and/or processing center set-up, compared to distributed processing, it can be easier to ensure data quality, and accomplish maintenance and updates to the calculation engine, compliance to data privacy regulations and/or troubleshooting.

While this specification contains many specific implementation details, these should not be construed as limitations on the scope of the disclosed technology or of what may be claimed, but rather as descriptions of features that may be specific to particular embodiments of particular disclosed technologies. Certain features that are described in this specification in the context of separate embodiments can also be implemented in combination in a single embodiment in part or in whole. Conversely, various features that are described in the context of a single embodiment can also be implemented in multiple embodiments separately or in any suitable subcombination. Moreover, although features may be described herein as acting in certain combinations and/or initially claimed as such, one or more features from a claimed combination can in some cases be excised from the combination, and the claimed combination may be directed to a subcombination or variation of a subcombination. Similarly, while operations may be described in a particular order, this should not be understood as requiring that such operations be performed in the particular order or in sequential order, or that all operations be performed, to achieve desirable results. Particular embodiments of the subject matter have been described. Other embodiments are within the scope of the following claims.

1. A method comprising:
receiving electronic health record (EHR) data;
determining, using a natural language processing (NLP) system, an instance of a personal health information (PHI) phrase in the received EHR data based at least in part on a NLP system confidence metric being above a threshold, wherein the confidence metric indicates the likelihood that the phrase is a PHI phrase, wherein the confidence metric is based at least in part on the PHI phrase appearing in a first context and wherein the confidence metric is higher in the first context than in another context, the first context being a location in the received EHR data where PHI is expected to be found;
determining another instance of the PHI phrase in the received EHR data, wherein the another instance of the PHI phrase does not have the same context as the first context but the another instance of the PHI phrase contains at least some of the same PHI as the instance of the PHI phrase;
removing the instance of the PHI phrase and the another instance of the PHI phrase from the received EHR data to produce cleaned EHR data based on the determining of the instance of the PHI phrase in the received EHR data and the determining of the another instance of the PHI phrase in the received EHR data; and
taking an action based on the cleaned EHR data
wherein the determining an instance of a PHI phrase, determining another instance of the PHI phrase, removing the instance of the PHI phrase and the another instance of the PHI phrase, and taking an action steps are performed in less than a minute for a plurality of EHR data files, each of the plurality of EHR data files having a data size of at least megabytes.

2. The method of claim 1, wherein a context of the another instance of the PHI phrase is such that the NLP system confidence metric based on a local context of the another instance is not above the threshold.

3. The method of claim 1, wherein the first context of the PHI phrase includes one or more of a group of prefixes comprising: Mr., Mrs., Ms., Miss, Dr., Doctor, Nurse, Signed by, signed by, Signed electronically by, signed electronically by, Electronically signed by, electronically signed by, Dear, dear, Name, name, NAME, Patient, patient, PATIENT, and RE:.

4. The method of claim 1, wherein a context of the another instance of the PHI phrase does not include one or more of a group of prefixes comprising: Mr., Mrs., Ms_{.,} Miss, Dr., Doctor, Nurse, Signed by, signed by, Signed electronically by, signed electronically by, Electronically signed by, electronically signed by, Dear, dear, Name, name, NAME, Patient, patient, PATIENT, and RE:.

5. The method of claim 1, wherein a context of the another instance of the PHI phrase includes at least one of literals, delimiters, and operators.

6. The method of claim 1, wherein determining, using a natural language processing (NLP) system, an instance of a personal health information (PHI) phrase comprises performing optical character recognition on image data contained in the EHR data.

7. The method of claim 1, wherein the PHI phrase is at least one of a person's names, disease, and medication.

8. (canceled)

9. A system comprising:
at least one programmable processor; and
a machine-readable medium storing instructions that, when executed by the at least one programmable processor, cause the at least one programmable processor to perform operations comprising:
receiving EHR data;
determining, using a natural language processing (NLP) system, an instance of a personal health information (PHI) phrase in the received EHR data based at least in part on a NLP system confidence metric being above a threshold, wherein the confidence metric indicates the likelihood that the phrase is a PHI phrase, wherein the confidence metric is based at least in part on the PHI phrase appearing in a first context and wherein the confidence metric is higher in the first context than in another context, the first context being a location in the received EHR data where PHI is expected to be found;
determining another instance of the PHI phrase in the received EHR data, wherein the another instance of the PHI phrase does not have the same context as the first context but the another instance of the PHI phrase contains at least some of the same PHI as the instance of the PHI phrase;
removing the instance of the PHI phrase and the another instance of the PHI phrase from the received EHR data to produce cleaned EHR data based on the determining of the instance of the PHI phrase in the received EHR data and the determining of the another instance of the PHI phrase in the received EHR data; and
taking an action based on the cleaned EHR data
wherein the determining an instance of a PHI phrase, determining another instance of the PHI phrase, removing the instance of the PHI phrase and the another instance of the PHI phrase, and taking an action steps are performed in less than a minute for a plurality of EHR data files, each of the plurality of EHR data files having a data size of at least megabytes.

10. The system of claim 9, wherein a context of the another instance of the PHI phrase is such that the NLP system confidence metric is not above the threshold.

11. The system of claim 9, wherein the first context of the PHI phrase includes one or more of a group of prefixes comprising: Mr., Mrs., Ms., Miss, Dr., Doctor, Nurse, Signed by, signed by, Signed electronically by, signed electronically by, Electronically signed by, electronically signed by, Dear, dear, Name, name, NAME, Patient, patient, PATIENT, and RE:.

12. The system of claim 9, wherein a context of the another instance of the PHI phrase does not include one or more of a group of prefixes comprising: Mr., Mrs., Ms., Miss, Dr., Doctor, Nurse, and Signed by, signed by, Signed electronically by, signed electronically by, Electronically signed by, electronically signed by, Dear, dear, Name, name, NAME, Patient, patient, PATIENT, and RE:.

13. The system of claim 9, wherein a context of the another instance of the PHI phrase includes at least one of literals, delimiters, and operators.

14. The system of claim 9, wherein determining, using an NLP system, an instance of a PHI phrase comprises performing optical character recognition on image data contained in the EHR data.

15. The system of claim 9, wherein the PHI phrase is at least one of a person's names, disease, and medication.

16. (canceled)

17. One or more non-transitory computer program products storing instructions that, when executed by at least one programmable processor, cause the at least one programmable processor to perform operations comprising:
receiving EHR data;
determining, using a natural language processing (NLP) system, an instance of a personal health information (PHI) phrase in the received EHR data based at least in part on a NLP system confidence metric being above a threshold, wherein the confidence metric indicates the likelihood that the phrase is a PHI phrase, wherein the confidence metric is based at least in part on the PHI phrase appearing in a first context and wherein the confidence metric is higher in the first context than in another context, the first context being a location in the received EHR data where PHI is expected to be found;
determining another instance of the PHI phrase in the received EHR data, wherein the another instance of the PHI phrase does not have the same context as the first context but the another instance of the PHI phrase contains at least some of the same PHI as the instance of the PHI phrase;
removing the instance of the PHI phrase and the another instance of the PHI phrase from the received EHR data to produce cleaned EHR data based on the determining of the instance of the PHI phrase in the received EHR data and the determining of the another instance of the PHI phrase in the received EHR data; and
taking an action based on the cleaned EHR data
wherein the determining an instance of a PHI phrase, determining another instance of the PHI phrase, removing the instance of the PHI phrase and the another instance of the PHI phrase, and taking an action steps are performed in less than a minute for a plurality of EHR data files, each of the plurality of EHR data files having a data size of at least megabytes.

18. The one or more non-transitory computer program products of claim 17, wherein a context of the another instance of the PHI phrase is such that the NLP system confidence metric is not above the threshold.

19. The one or more non-transitory computer program products of claim 17, wherein the PHI phrase is at least one of a person's names, disease, and medication.

20. The one or more non-transitory computer program products of claim 17, wherein the first context of the PHI phrase includes one or more of a group of prefixes comprising: Mr., Mrs., Ms., Miss, Dr., Doctor, Nurse, Signed by, signed by, Signed electronically by, signed electronically by, Electronically signed by, electronically signed by, Dear, dear, Name, name, NAME, Patient, patient, PATIENT, and RE:.
